(19) United States
(12) Patent Application Publication
(10) Pub. No.: US 2024/0325051 A1
(43) Pub. Date: Oct. 3, 2024

(54) SAFETY LANCET TRANSSEPTAL NEEDLE

(71) Applicant: Boston Scientific Scimed, Inc., Maple Grove, MN (US)

(72) Inventors: Spencer Fodness-Bondhus, Columbia Heights, MN (US); Edward Louis Gonda, Minneapolis, MN (US)

(21) Appl. No.: 18/615,229

(22) Filed: Mar. 25, 2024

Related U.S. Application Data
(60) Provisional application No. 63/492,541, filed on Mar. 28, 2023.

(51) Int. Cl. A61B 17/34; A61B 17/00

(52) U.S. Cl. CPC A61B 17/3496; A61B 17/00234; A61B 2017/00247; A61B 2017/00292; A61B 2017/00367; A61B 2017/00862

(57) ABSTRACT

A safety lancet transseptal access system is disclosed. The system includes a housing having a proximal end and a distal end, the proximal end having an opening with a plunger and the distal end terminating in a housing wall, a housing lumen extending through the housing and a release mechanism coupled to the housing. The system further includes a puncture device spring connected to the plunger and a disk. The system further includes a puncture device connected to the disk. When the plunger is pushed towards the distal end of the housing, the puncture device spring compresses triggering the release mechanism which then causes the puncture device to be deployed past the housing wall. Additionally, once deployed, tension from the puncture device spring pulls the disk and the puncture device back into the housing to a neutral position.

### CROSS REFERENCE TO RELATED APPLICATIONS

This application claims the benefit of U.S. Provisional Application No. 63/492,541 entitled, “SAFETY LANCET TRANSSEPTAL NEEDLE,” filed Mar. 28, 2023, which is referenced herein in its entirety.

### TECHNICAL FIELD

The present invention relates generally to devices and methods for transseptal access systems. More specifically, the present invention is concerned with a safety lancet transseptal perforation device providing access to a patient's heart.

### BACKGROUND

Devices currently exist for creating a puncture, channel, or perforation within a tissue located in a body of a patient. One such device is the Brockenbrough™ Needle, which is commonly used to puncture the atrial septum of the heart. This device is a stiff elongated needle, which is structured such that it may be introduced into a body of the patient via the femoral vein and directed towards the heart. This device relies on the use of mechanical force to drive the sharp tip through the septum.

Alternatively, radiofrequency perforation apparatuses have been developed, whereby the septal perforation is accomplished by the application of focused radiofrequency energy to the septal tissue via an electrode at the distal end of a relatively thin conductive probe. These devices are complex and costly.

Against this background, there exists a continuing need in the industry to provide improved transseptal access system devices and methods. An object of the present invention is therefore to provide such a transseptal access system.

### SUMMARY

In Example 1, a safety lancet transseptal access system includes a housing having a proximal end and a distal end, the proximal end having an opening with a plunger and the distal end terminating in a housing wall, a housing lumen extending through the housing and a release mechanism coupled to the housing; a puncture device spring connected to the plunger and a disk; and a puncture device connected to the disk.

Example 2 is the safety lancet of Example 1, wherein when the plunger is pushed towards the distal end of the housing, the puncture device spring compresses triggering the release mechanism which then causes the puncture device to be deployed past the housing wall.

Example 3 is the safety lancet of any of Examples 1-2, wherein once deployed, tension from the puncture device spring pulls the disk and the puncture device back into the housing to a neutral position.

Example 4 is the safety lancet of any of Examples 1-3, wherein the housing further comprises a plunger return spring.

Example 5 is the safety lancet of Example 4, wherein the plunger return spring is configured to push the plunger back to an undeployed state once deployed.

Example 6 is the safety lancet of any of Examples 1-5, wherein the puncture device may be redeployed.

Example 7 is the safety lancet of Example 1, wherein the safety lancet further comprises a tubular dilator having an elongated body with a proximal end portion connected to the housing wall and a distal end portion terminating in a distal tip, a dilator lumen extending through the body, the body being dimensioned to slidingly receive a hypotube and the puncture device.

Example 8 is the safety lancet of Example 7, wherein the hypotube may be extended out of the distal tip of the dilator by the puncture device spring.

Example 9 is the safety lancet of Example 1, wherein the puncture device spring causes the puncture device to perforate an atrial septum and subsequently withdraw, allowing the hypotube and the dilator to advance through the atrial septum.

Example 10 is the safety lancet of any of Examples 1-9, wherein the hypotube operates to gradually enlarge a perforation initially created by the puncture device.

Example 11 is the safety lancet of Example 1, wherein the puncture device is an automatic spring-loaded puncture device.

Example 12 is the safety lancet of Example 1, wherein the puncture device is mechanically triggered by a user applying an outside force.

Example 13 is the safety lancet of Example 1, wherein the puncture device may be any device that can pierce the atrial septum and withdraw.

Example 14 is the safety lancet of Example 1, wherein the release mechanism may be made of any sufficiently durable and stiff plastic or metal, among other materials.

Example 15 is the safety lancet of Example 1, wherein the disk may be threaded, glued, or welded to the puncture device.

In Example 16, a safety lancet transseptal access system includes a housing having a proximal end and a distal end, the proximal end having an opening with a plunger and the distal end terminating in a housing wall, a housing lumen extending through the housing and a release mechanism coupled to the housing; a puncture device spring connected to the plunger and a disk; and a puncture device connected to the disk; wherein when the plunger is pushed towards the distal end of the housing, the puncture device spring compresses triggering the release mechanism which then causes the puncture device to be deployed past the housing wall, and wherein once deployed, tension from the puncture device spring pulls the disk and the puncture device back into the housing to a neutral position.

Example 17 is the safety lancet of Example 16, wherein the housing further comprises a plunger return spring and wherein the plunger return spring is configured to push the plunger back to an undeployed state once deployed.

Example 18 is the safety lancet of Example 17, wherein the puncture device may be redeployed.

Example 19 is the safety lancet of Example 16, wherein the safety lancet further comprises a tubular dilator having an elongated body with a proximal end portion connected to the housing wall and a distal end portion terminating in a distal tip, a dilator lumen extending through the body, the body being dimensioned to slidingly receive a hypotube and the puncture device.

Example 20 is the safety lancet of claim 19, wherein the hypotube may be extended out of the distal tip of the dilator by the puncture device spring.

Example 21 is the safety lancet of Example 16, wherein the puncture device spring causes the puncture device to perforate an atrial septum and subsequently withdraw, allowing the hypotube and the dilator to advance through the atrial septum.

Example 22 is the safety lancet of Example 20, wherein the hypotube operates to gradually enlarge a perforation initially created by the puncture device.

Example 23 is the safety lancet of Example 16, wherein the puncture device is an automatic spring-loaded puncture device.

Example 24 is the safety lancet of Example 16, wherein the puncture device is mechanically triggered by a user applying an outside force.

Example 25 is the safety lancet of claim 16, wherein the puncture device may be any device that can pierce the atrial septum and withdraw.

In Example 26, a safety lancet transseptal access system includes a housing having a proximal end and a distal end, the proximal end having an opening with a plunger and the distal end terminating in a housing wall, a housing lumen extending through the housing and a release mechanism coupled to the housing; a puncture device spring connected to the plunger and a disk; and a tubular dilator having an elongated body having a proximal end portion connected to the housing wall and a distal end portion terminating in a distal tip, a dilator lumen extending through the body, the body being dimensioned to slidingly receive a hypotube and a puncture device, the puncture device being connected to the disk; wherein when the plunger is pushed towards the distal end of the housing, the puncture device spring compresses triggering the release mechanism which then causes the puncture device to be deployed past the housing wall, and wherein once deployed, tension from the puncture device spring pulls the disk and the puncture device back into the housing to a neutral position.

Example 27 is the safety lancet of Example 26, wherein the housing further comprises a plunger return spring and wherein the plunger return spring is configured to pull the plunger back to an undeployed state once deployed.

Example 28 is the safety lancet of Example 26, wherein the puncture device and the hypotube may be redeployed.

Example 29 is the safety lancet of Example 26, wherein the hypotube may be extended out of the distal tip of the dilator by the puncture device spring.

Example 30 is the safety lancet of Example 26, wherein the puncture device is an automatic spring-loaded puncture device.

Example 31 the safety lancet of Example 26, wherein the puncture device is mechanically triggered by a user by applying an outside force.

Example 32 is the safety lancet of Example 26, wherein the puncture device may be any device that can pierce the atrial septum and withdraw.

In Example 33, a method of making a safety lancet transseptal access system, includes: providing a housing having a proximal end and a distal end, the proximal end having an opening with a plunger and the distal end terminating in a housing wall, a housing lumen extending through the housing and a release mechanism coupled to the housing; securing a puncture device spring connected to the plunger and a disk; and a puncture device connected to the disk; wherein when the plunger is pushed towards the distal end of the housing, the puncture device spring compresses triggering the release mechanism which then causes the puncture device to be deployed past the housing wall, and wherein once deployed, tension from the puncture device spring pulls the disk and the puncture device back into the housing to a neutral position.

Example 34 is the safety lancet of Example 33, wherein the housing further comprises a plunger return spring.

Example 35 the safety lancet of Example 33, wherein the puncture device may be redeployed

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIGS. 1A-1D are schematic illustrations of a medical procedure within a patient's heart utilizing a transseptal access system according to embodiments of the present disclosure.

FIG. 2 is a schematic illustration of a dilator having a puncture device of the transseptal access system illustrated in FIG. 1, according to embodiments of the present disclosure.

FIGS. 3A-3E are schematic illustrations of a safety lancet mechanism built into a dilator having a puncture device of the transseptal access system illustrated in FIG. 1, according to embodiments of the present disclosure.

FIGS. 4A-4G are schematic illustrations of a multi-deployment safety lancet mechanism built into a dilator having a puncture device and a hypotube of the transseptal access system illustrated in FIG. 1, according to embodiments of the present disclosure.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

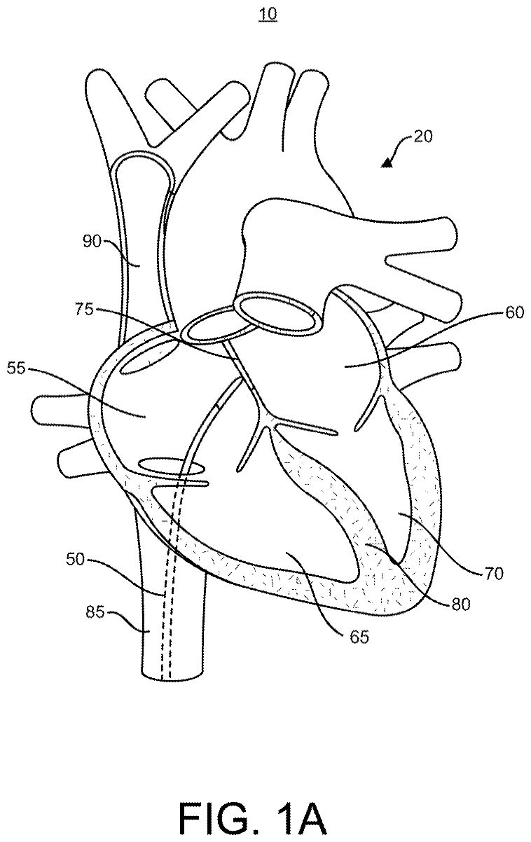

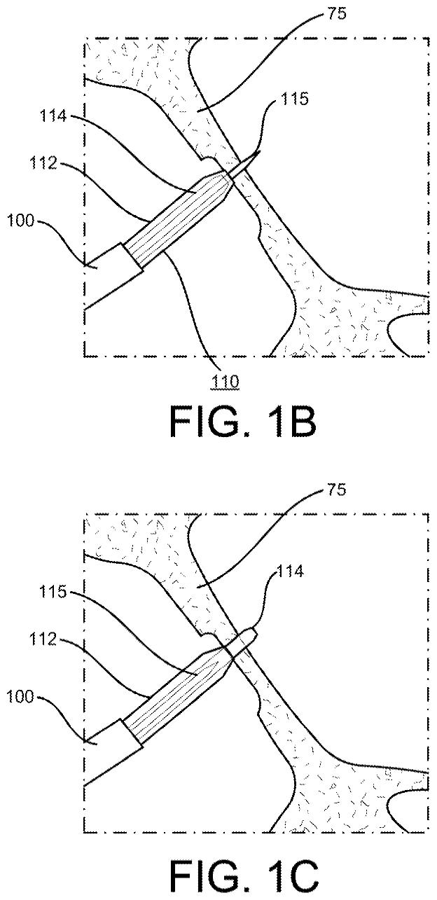

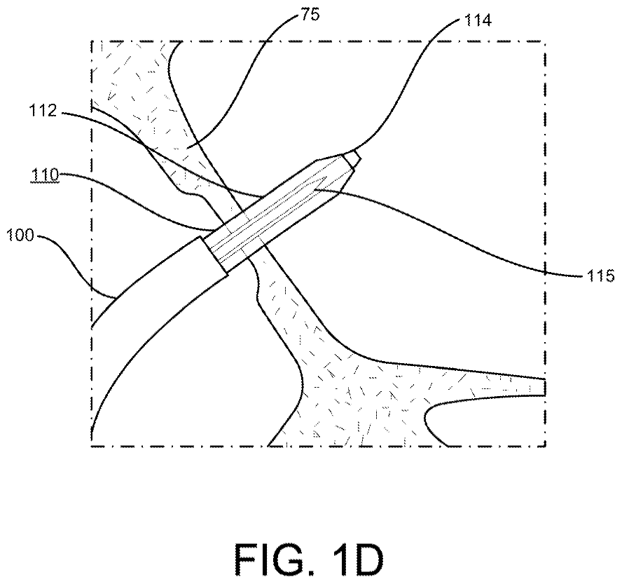

FIGS. 1A-1D are schematic illustrations of a medical procedure 10 within a patient's heart 20 utilizing a transseptal access system 50 according to embodiments of the disclosure. As is known, the human heart 20 has four chambers, a right atrium 55, a left atrium 60, a right ventricle 65 and a left ventricle 70. Separating the right atrium 55 and the left atrium 60 is an atrial septum 75, and separating the right ventricle 65 and the left ventricle 70 is a ventricular septum 80. As is further known, deoxygenated blood from the patient's body is returned to the right atrium 55 via an inferior vena cava (IVC) 85 or a superior vena cava (SVC) 90.

Various medical procedures have been developed for diagnosing or treating physiological ailments originating within the left atrium 60 and associated structures. Exemplary such procedures include, without limitation, deployment of diagnostic or mapping catheters within the left atrium 60 for use in generating electroanatomical maps or diagnostic images thereof. Other exemplary procedures include endocardial catheter-based ablation (e.g., radiofrequency ablation, pulsed field ablation, cryoablation, laser ablation, high frequency ultrasound ablation, and the like) of target sites within the chamber or adjacent vessels (e.g., the pulmonary veins and their ostia) to terminate cardiac arrythmias such as atrial fibrillation and atrial flutter. Still other exemplary procedures may include deployment of left atrial appendage (LAA) closure devices. Of course, the foregoing examples of procedures within the left atrium 60 are merely illustrative and in no way limiting with respect to the present disclosure.

The medical procedure 10 illustrated in FIG. 1 is an exemplary embodiment for providing access to the left atrium 60 using the transseptal access system 50 for subsequent deployment of the aforementioned diagnostic and/or therapeutic devices within the left atrium 60. In embodiments, as shown in FIGS. 1B-1D, the transseptal access system 50 may include a mechanical perforation device. In other embodiments, the transseptal access system 50 may include a radiofrequency perforation device. As shown in FIG. 1, target tissue site can be defined by tissue on the atrial septum 75. In the illustrated embodiment, the target site is accessed via the IVC 85, for example through the femoral vein, according to conventional catheterization techniques. In other embodiments, access to the target site on the atrial septum 75 may be accomplished using a superior approach wherein the transseptal access system 50 is advanced into the right atrium 55 via the SVC 90.

In the illustrated embodiment, the transseptal access system 50 includes an introducer sheath 100 and a dilator 110. The dilator 110 includes a dilator body 112 terminating in a distal tip, a hypotube 114 and a mechanical puncture device 115. As shown, in the assembled use state illustrated in FIGS. 1B-1D, the hypotube 114 and the puncture device 115 can be disposed within the dilator body 112, which itself can be disposed within the sheath 100. In one embodiment in which the transseptal access system 50 is deployed into the right atrium 55 via the IVC 85, a user introduces a guidewire (not shown) into a femoral vein, typically the right femoral vein, and advances it towards the heart 20. The sheath 100 may then be introduced into the femoral vein over the guidewire, and advanced towards the heart 20. In one embodiment, the distal ends of the guidewire and sheath 100 are then positioned in the SVC 90. These steps may be performed with the aid of an imaging system, e.g., fluoroscopy or ultrasonic imaging. The dilator 110 may then be introduced into the sheath 100 and over the guidewire, and advanced through the sheath 100 into the SVC 90. Alternatively, the dilator 110 may be fully inserted into the sheath 100 prior to entering the body, and both may be advanced simultaneously towards the heart 20. When the guidewire, sheath 100 and dilator 110 have been positioned in the SVC 90, the guidewire is removed from the body, and the sheath 100 and the dilator 110 are retracted so that their distal ends are positioned in the right atrium 55. The hypotube 114 and the puncture device 115 described can then be introduced into the dilator 110, and advanced towards the heart 20. In certain embodiments, the dilator 110 may be introduced into the body without a need for the sheath 100. In other embodiments, the dilator 110 may include only the puncture device 115 with no hypotube.

In embodiments, the user may position the distal tip of the dilator 110 against the atrial septum 75, which can be done under imaging guidance. In embodiments, the hypotube 114 and the puncture device 115 are positioned at a safe distance inside the dilator body 112 proximal to the distal tip so as to prevent accidental puncturing of the atrial septum 75. The dilator 110 may be dragged along the atrial septum 75 and positioned, for example, against the fossa ovalis of the atrial septum 75 under imaging guidance. A variety of additional steps may be performed, such as measuring one or more properties of the target site, for example an electrogram or ECG (electrocardiogram) tracing and/or a pressure measurement, or delivering material to the target site, for example delivering a contrast agent. Such steps may facilitate the localization of the dilator body 110 at the desired target site.

As demonstrated in FIGS. 1B-1D, with the dilator 110 positioned at the target site, the user may trigger the puncture device 115 to advance outside of the dilator body 112, distal to the distal tip, and mechanically pierce the atrial septum 75. In embodiments, the puncture device 115 may be an automatic spring-loaded puncture device. In other embodiments, the user may mechanically trigger the puncture device 115 by applying an outside force. Once the atrial septum 75 is perforated, the pressure applied may then force the hypotube 114 to extend outside of the dilator body 112 and across the atrial septum 75 in order to widen the perforation initially created by the puncture device 115. Additionally, as the hypotube 114 extends outside of the dilator body 112, the puncture device 115 auto-retracts into the dilator body 112 to prevent further tissue interaction. With the atrial septum 75 pierced by the puncture device 115 and enlarge by the hypotube 114, the dilator 110 may then advance forward into the left atrium 60.

The present disclosure describes novel devices and methods for providing safe transseptal access to the left atrium 60 using a mechanical safety lancet transseptal perforation device. As will be explained in greater detail herein, the embodiments of the present disclosure simplify the means of providing a puncture device during medical procedures within a patient's heart utilizing a transseptal access system.

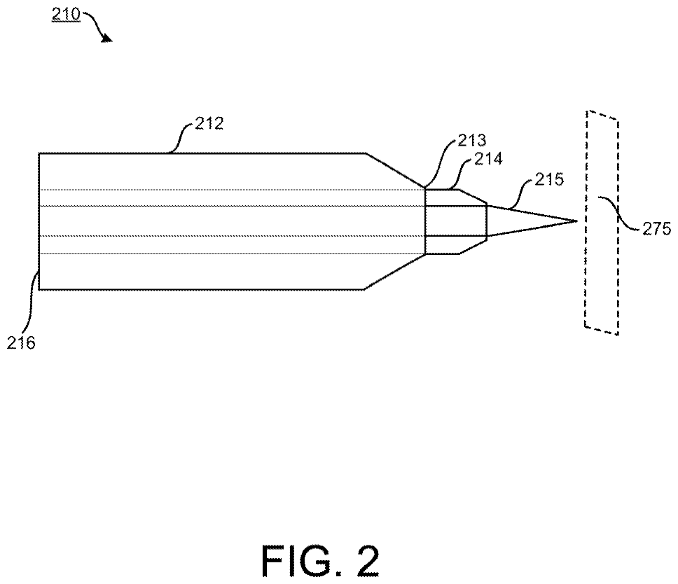

FIG. 2 is a schematic illustration of a dilator 210 with a puncture device 215 of the transseptal access system 50 illustrated in FIG. 1. As shown, the tubular dilator 210 includes an elongated body 212 having a proximal end portion and a distal end portion terminating in a distal tip 213. The dilator 210 also includes a dilator lumen 216 extending longitudinally through the dilator body 212. In addition, a hypotube 214 and the puncture device 215 are positioned within the dilator lumen 216. The hypotube 214 and the puncture device 215 may move longitudinally proximal and distal to the distal tip 213 of the dilator body 212 as needed. In embodiments, the hypotube 214 and the puncture device 215 move slidingly proximal and distal to the distal tip 213 of the dilator body 212 to facilitate in puncturing the atrial septum 275 of a patient's heart.

In embodiments, the distal tip 213 of the dilator 210 is positioned on the atrial septum 275, with the hypotube 214 and the puncture device 215 positioned at a safe distance inside the dilator body 212 proximal to the distal tip 213 so as to prevent accidental puncturing of the atrial septum 275. In embodiments, a user may trigger the puncture device 215 to advance outside of the dilator body 212, distal to the distal tip 213, in order to pierce the atrial septum 275. In embodiments, the puncture device 215 may be an automatic spring-loaded puncture device. In other embodiments, the user may mechanically trigger the puncture device 215 by applying an outside force. In embodiments, once the atrial septum 275 is perforated, the continued pressure applied may then trigger the hypotube 214 to advance outside of the dilator body 212 and across the atrial septum 275, while the puncture device 215 auto-retracts into the dilator body 212 proximal to the distal tip 213 to prevent further tissue interaction. In embodiments, the hypotube 214 operates to gradually enlarge the perforation initially created by the puncture device 215 to permit the advancement of the dilator 210 across the atrial septum 275 and into the left atrium in order to proceed with the medical procedure. In embodiments, a sheath (not shown) may be advanced through the dilator 210 for subsequent deployment of diagnostic and/or therapeutic devices within the left atrium 60 of FIG. 1, as mentioned above.

In embodiments, the puncture device 215 may be any device that can pierce the septum 275 and quickly withdraw, examples include a stylet, a needle, a hypotube, among others. The piercing of the atrial septum 275 and quick withdrawal of the puncture device 215 ensures that the sharp tip of the puncture device 215 does not cause trauma to the tissue. The puncture device 215 also facilitates in crossing the atrial septum 275 better than a typical lancet needle. Furthermore, the puncture device of the present invention also reduces safety concerns in using a sharp crossing element and eliminates the risk of coring. In embodiments, the hypotube 214 may have a beveled, as shown, or round leading edge to further mitigate safety concerns, limit skiving, and assist in crossing the septum. In other embodiments, the dilator 210 may pierce the atrial septum 275 without the need for a hypotube.

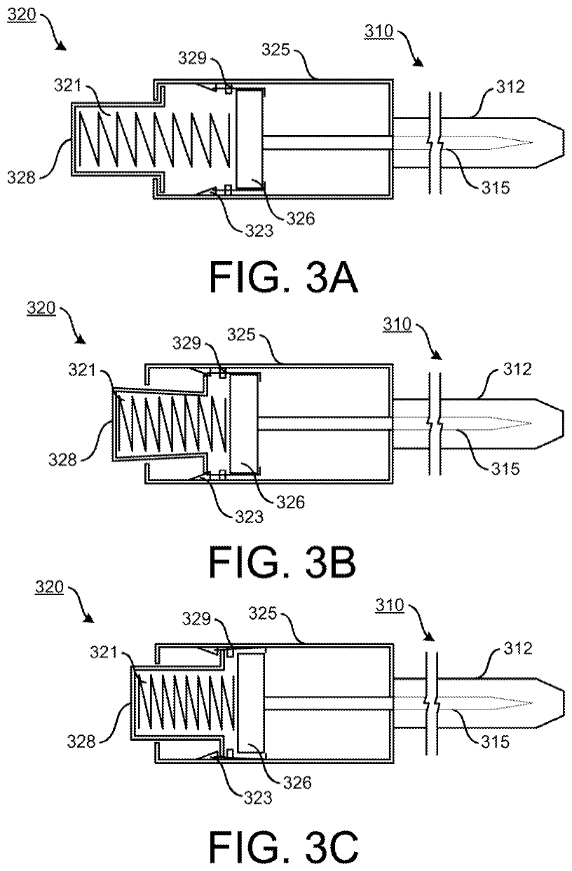

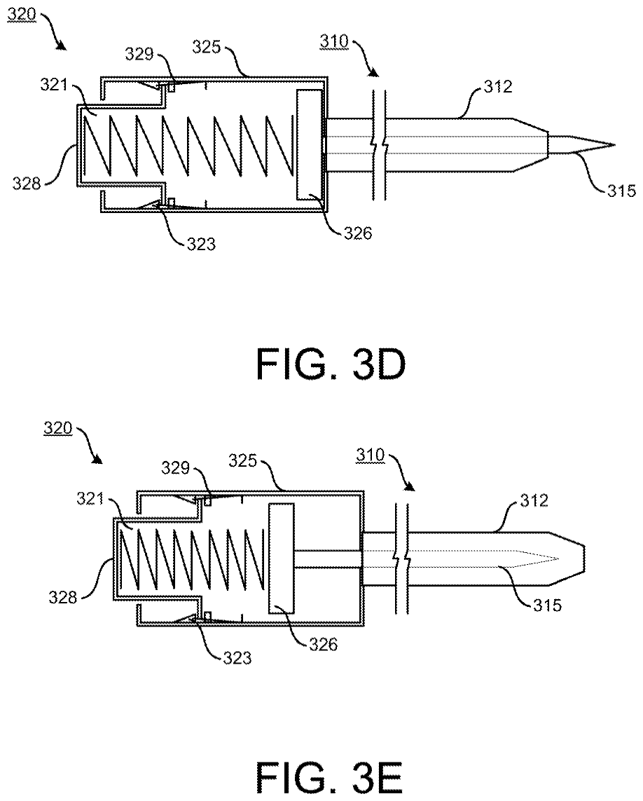

FIGS. 3A-3E are schematic illustrations of a safety lancet transseptal perforation device 320 built into a dilator 310 of the transseptal access system 50 of FIG. 1, according to embodiments of the present disclosure. As shown, the typical safety lancet transseptal perforation device 320 includes a lancet housing 325 having a proximal end portion and a distal end portion. The housing 325 also includes a housing lumen extending longitudinally through the housing 325. The distal end portion of the housing 325 terminates in a housing wall. The proximal end portion of the housing 325 includes an opening having a plunger 328, a ramp 323 placed on the top and bottom of the housing 325 inside the housing lumen, a disk 326, and a needle release mechanism 329 connected to the ramp 323 placed on the top and bottom of the housing lumen and enclosing the disk 326. In embodiments, the needle release mechanism 329 may be stiff and pinned to the ramp 323 or flexible and attached to the ramp 323. In embodiments, the needle release mechanism 329 may be made of any sufficiently durable and stiff plastic or metal, among other materials. In embodiments, the distal portion of the disk 326 of the safety lancet transseptal perforation device 320 is connected to a puncture device 315 of the dilator 310. In embodiments, the distal portion of the disk 326 of the safety lancet transseptal perforation device 320 may be threaded, glued, or welded to the puncture device 315 of the dilator 310.

As shown, in the proximal end portion of the housing 325, the plunger 328 includes a puncture device spring 321 that is connected to the proximal portion of the disk 326 and facilitates in the movement of the disk 326 and therefore the puncture device 315 when compressed. In embodiments, the housing wall acts as a stop for the disk 326 so that the disk 326 may not advance past the housing wall. However, in embodiments, the puncture device 315 connected to the disk 326 may move through the housing wall as needed. In embodiments, the housing 325 may vary in dimension. In some embodiments, generally, the housing 325 may be about 10 mm in width, 15 mm in length, and 5 mm in height. In other embodiments, the housing 325 may be enlarged for handling, improved ability to generate larger forces, among other reasons, and be up to 5 cm in width, 15 cm in length, and 5 cm in height.

Furthermore, in embodiments, the tubular dilator 310 includes an elongated dilator body 312 having a proximal end portion and a distal end portion terminating in a distal tip. The dilator 310 also includes a dilator lumen extending longitudinally through the dilator body 312. In embodiments, the puncture device 315 is positioned within the dilator lumen. In embodiments, as shown, the proximal end portion of the dilator body 312 is placed on the housing wall of the safety lancet transseptal perforation device 320. In further embodiments, the puncture device 315 is connected to the disk 326 of the safety lancet transseptal perforation device 320. The puncture device 315 may move longitudinally proximal and distal to the distal tip of the dilator body 312 as needed, and its movement may be controlled by the puncture device spring 321 connected to the disk 326. In embodiments, the puncture device 315 moves slidingly proximal and distal to the distal tip of the dilator body 312 to facilitate in puncturing the atrial septum 75 of FIG. 1 of a patient's heart.

In embodiments, a series of steps, FIGS. 3A-3E, trigger the safety lancet release mechanism 329 to advance the puncture device 315 outside the dilator body 312 to pierce the atrial septum and subsequently back to a neutral point inside the dilator body 312. As shown in FIG. 3A, the safety lancet mechanism begins in a default undeployed state wherein the puncture device spring 321 is relaxed, and the disk 326 is positioned near the ramp 323 inside the housing 325. In embodiments, while in an undeployed state, the distal tip of the dilator body 312 is positioned on the atrial septum, with the puncture device 315 positioned at a safe distance inside the dilator body 312 proximal to the distal tip so as to prevent accidental puncturing of the atrial septum. As shown in FIG. 3B, a user may press the plunger 328 towards the distal end portion of the housing 325 causing the puncture device spring 321 to compress and the plunger 328 to begin moving towards the ramp 323. In embodiments, the puncture device 315 is still positioned at a safe distance inside the dilator body 312 proximal to the distal tip so as to prevent accidental puncturing of the atrial septum.

As shown in FIG. 3C, as the user continues to press the plunger 328 into the distal end portion of the housing 325, the puncture device spring 321 is fully compressed. This compression and force towards the distal end portion of the housing 325 causes the plunger 328 to advance over the ramp 323 located on the top and bottom of the housing 325 inside the housing lumen. This move forward further causes the plunger 328 to snap into a retention zone, triggering the needle release mechanism 329. In embodiments, the puncture device 315 is still positioned at a safe distance inside the dilator body 312 proximal to the distal tip so as to prevent accidental puncturing of the atrial septum. As shown in FIG. 3D, once the needle release mechanism 329 is triggered, the pressure caused by the compression of the puncture device spring 321 drives the disk 326 forward at a high speed towards the housing wall. In embodiments, this momentum also carries the puncture device 315 past its neutral point, causing it to move outside of the dilator body 312, distal to the distal tip, and places the puncture device spring 321 in tension. As the disk 326 simultaneously reaches the housing wall, the disk is stopped by the housing wall and is prevented from moving any further. Finally, as shown in FIG. 3E, in embodiments, the tension created in the puncture device spring 321 pulls the disk 326, and therefore the puncture device 315, back into the housing 325 and the puncture device spring 321 comes to a rest. Additionally, the puncture device 315 is again positioned at a safe distance inside the dilator body 312 proximal to the distal tip so as to prevent further puncturing of the atrial septum. In a typical safety lancet mechanism, because the plunger 328 is captured, the puncture device 315 cannot be redeployed outside of the dilator body 312.

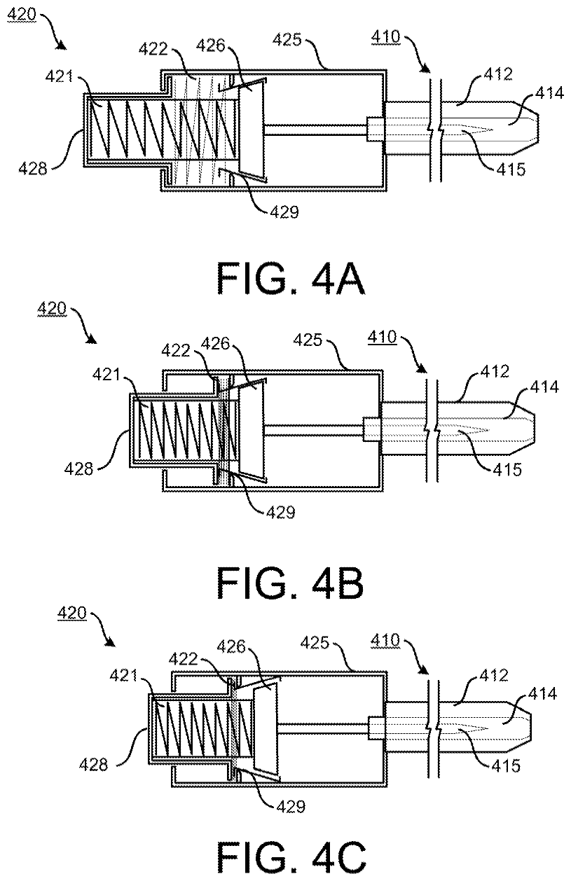

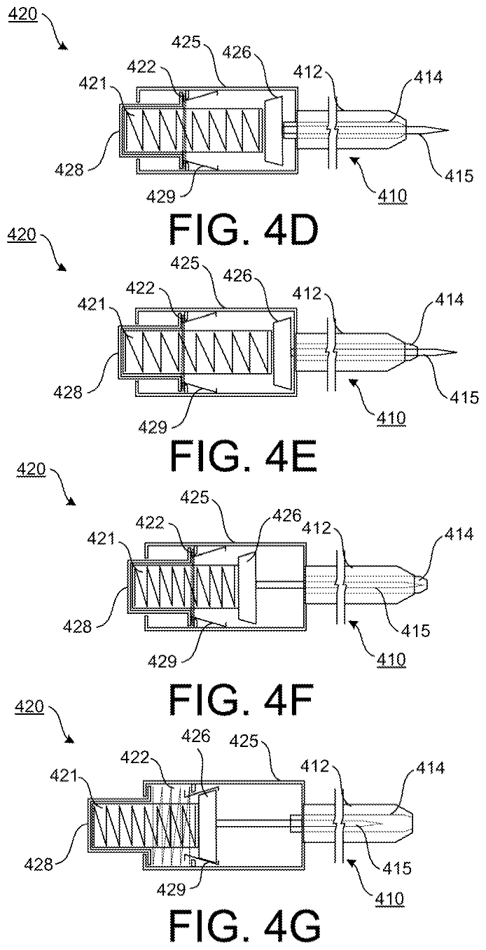

FIGS. 4A-4G are schematic illustrations of a multi-deployment safety lancet transseptal perforation device 420 built into a dilator 410 of the transseptal access system 50 of FIG. 1. As shown, the multi-deployment safety lancet transseptal perforation device 420 includes a lancet housing 425 having a proximal end portion and a distal end portion. The housing 425 further includes a housing lumen extending longitudinally through the housing 425. The distal end portion of the housing 425 terminates in a housing wall. The proximal end portion of the housing 425 includes a plunger 428, a disk 426, a needle release mechanism 429 placed on the top and bottom of the housing lumen and enclosing the disk, a puncture device spring 421, and a plunger return spring 422. In embodiments, the needle release mechanism 429 may be made of any sufficiently durable and stiff plastic or metal, among other materials. In embodiments, the distal portion of the disk 426 of the safety lancet transseptal perforation device 420 is connected to a puncture device 415 of the dilator 410. In embodiments, the distal portion of the disk 426 of the safety lancet transseptal perforation device 420 may be threaded, glued, or welded to the puncture device 415 of the dilator 410.

As shown, in the proximal end portion of the housing 425, the puncture device spring 421 is connected to the proximal portion of the disk 426 and facilitates in the movement of the disk 426 and therefore the puncture device 415. In embodiments, the plunger return spring 422 is constrained by the plunger 428 and the housing 425. In embodiments, the housing wall acts as a stop for the disk 426 so that the disk 426 may not advance past the housing wall. However, in embodiments, the puncture device 415 connected to the disk 426 may move through the housing wall as needed. In embodiments, the housing 425 may vary in dimension. In some embodiments, generally, the housing 425 may be about 10 mm in width, 15 mm in length, and 5 mm in height. In other embodiments, the housing 425 may be enlarged for handling, improved ability to generate larger forces, among other reasons, and be dimensioned up to 5 cm in width, 15 cm in length, and 5 cm in height. In embodiments, the plunger return spring 422 functions to return the plunger 428 to an initial undeployed state so that the safety lancet mechanism may be redeployed. In embodiments, the plunger 428 is free to move in and out of the housing lumen based on the forces applied to it by the puncture device spring 421, the plunger return spring 422 and the user. More specifically, in embodiments, the plunger 428 may move into the housing lumen and subsequently out of the lumen, to be redeployed, based on the forces applied to the plunger return spring 422.

Furthermore, in embodiments, the dilator 410 may be substantially structurally and functionally identical to the dilator 210 in FIG. 2. In embodiments, as shown, the tubular dilator 410 includes an elongated dilator body 412 having a proximal end portion and a distal end portion terminating in a distal tip. The dilator 410 also includes a dilator lumen extending longitudinally through the dilator body 412. In embodiments, a hypotube 414 and the puncture device 415 are positioned within the dilator lumen. In embodiments, as shown, the proximal end portion of the dilator body 412 is placed on the housing wall of the safety lancet transseptal perforation device 420. In further embodiments, the puncture device 415 is connected to the disk 426 of the safety lancet transseptal perforation device 420. The hypotube 414 and the puncture device 415 may move longitudinally proximal and distal to the distal tip of the dilator body 412 as needed. In embodiments, the movements of the hypotube 414 and the puncture device 415 may be controlled by the puncture device spring 321 that is connected to the disk 326. In embodiments, the hypotube 414 and the puncture device 415 move slidingly proximal and distal to the distal tip of the dilator body 418 to facilitate in puncturing and enlarging the atrial septum 75 and 275 of FIGS. 1 and 2 of a patient's heart. In other embodiments, the dilator 410 may pierce the atrial septum without the need for a hypotube.

In embodiments, a series of steps, FIGS. 4A-4G, trigger the puncture device spring 421 in the multi-deployment safety lancet transseptal perforation device 420 to cause the puncture device 415 of the dilator 410 to puncture the atrial septum of a patient's heart and subsequently withdraw, allowing the hypotube 414 and dilator 410 to advance through the atrial septum. Additionally, in embodiments, since the plunger return spring 422 functions to return the plunger 428 back to an initial undeployed state, the safety lancet mechanism may be redeployed in order to puncture the atrial septum again. As shown in FIG. 4A, the safety lancet device 420 begins in a default undeployed state wherein the puncture device spring 421 and plunger return spring 422 are in a relaxed position. In embodiments, while in an undeployed state, the distal tip of the dilator body 412 is positioned on the atrial septum, with the hypotube 414 and the puncture device 415 positioned at a safe distance inside the dilator body 412 proximal to the distal tip so as to prevent accidental puncturing of the atrial septum.

As shown in FIG. 4B, a user may press the plunger 428 towards the distal end portion of the housing 425 causing the puncture device spring 421 and the plunger return spring 422 to compress. In embodiments, as the plunger 428 is pushed forward, the plunger 428 will make initial contact with the release mechanism 429. In embodiments, the hypotube 414 and the puncture device 315 are still positioned at a safe distance inside the dilator body 412 proximal to the distal tip so as to prevent accidental puncturing of the atrial septum. As shown in FIG. 4C, as the user continues to press the plunger 428 into the distal end portion of the housing 425, the puncture device spring 421 and the plunger return spring 422 become fully compressed. This compression and force towards the distal end portion of the housing 425 causes the release mechanism 429 to continue to advance.

In embodiments, as shown in FIG. 4D, once the release mechanism 429 is triggered, while the plunger return spring 422 is still fully compressed, the pressure caused by the compression of the puncture device spring 421 drives the disk 426 past its neutral point at a high speed towards the housing wall. In embodiments, the disk 426 is prevented from moving any further because of the housing wall. In further embodiments, the momentum from the disk 426 also carries the puncture device 415 past its neutral point, causing it to move outside of the dilator body 412, distal to the distal tip, in order to pierce the atrial septum while the hypotube 414 continues to be positioned at a safe distance inside the dilator body 412 proximal to the distal tip. In embodiments, the push forward also places the puncture device spring 321 in tension. In embodiments, the puncture device 415 may be an automatic spring-loaded puncture device. In other embodiments, the user may mechanically trigger the puncture device 415 by applying an outside force.

As shown in FIG. 4E, after the puncture device 415 has punctured the atrial septum, the hypotube 414 is automatically advanced by the same release mechanism and momentum outside of the dilator body 412, distal to the distal tip, in order to enlarge the perforation initially created by the puncture device 415, if needed. In embodiments, as shown in FIG. 4F, tension from the puncture device spring 421 pulls the disk 426 back into the housing 425 where the puncture device spring 421 and disk 426 come to rest. Furthermore, because of this, the puncture device 415 simultaneously auto-retracts into the dilator body 412. Finally, as shown in FIG. 4G, the plunger return spring 422 pushes the plunger 428 back to its starting position towards the proximal end portion of the housing 425, with the puncture device spring 421 and plunger return spring 422 back to a relaxed undeployed state once again. This step also re-engages the needle release mechanism 429. In embodiments, the needle release mechanism 429 may be re-engaged by having its own separate spring to return to an initial undeployed position, or in other embodiments, the disk 426 may pull the needle release mechanism 429 back to its initial position. In embodiments, the user may redeploy the puncture device 415 if the transseptal crossing was not successful or continue advancing the dilator 410 across the atrial septum and remove the puncture device 415 and/or the hypotube 414 as needed.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A safety lancet transseptal access system comprising:
a housing having a proximal end and a distal end, the proximal end having an opening with a plunger and the distal end terminating in a housing wall, a housing lumen extending through the housing and a release mechanism coupled to the housing;
a puncture device spring connected to the plunger and a disk; and
a puncture device connected to the disk;
wherein when the plunger is pushed towards the distal end of the housing, the puncture device spring compresses triggering the release mechanism which then causes the puncture device to be deployed past the housing wall, and
wherein once deployed, tension from the puncture device spring pulls the disk and the puncture device back into the housing to a neutral position.

2. The safety lancet of claim 1, wherein the housing further comprises a plunger return spring and wherein the plunger return spring is configured to push the plunger back to an undeployed state once deployed.

3. The safety lancet of claim 2, wherein the puncture device may be redeployed.

4. The safety lancet of claim 1, wherein the safety lancet further comprises a tubular dilator having an elongated body with a proximal end portion connected to the housing wall and a distal end portion terminating in a distal tip, a dilator lumen extending through the body, the body being dimensioned to slidingly receive a hypotube and the puncture device.

5. The safety lancet of claim 4, wherein the hypotube may be extended out of the distal tip of the dilator by the puncture device spring.

6. The safety lancet of claim 1, wherein the puncture device spring causes the puncture device to perforate an atrial septum and subsequently withdraw, allowing the hypotube and the dilator to advance through the atrial septum.

7. The safety lancet of claim 5, wherein the hypotube operates to gradually enlarge a perforation initially created by the puncture device.

8. The safety lancet of claim 1, wherein the puncture device is an automatic spring-loaded puncture device.

9. The safety lancet of claim 1, wherein the puncture device is mechanically triggered by a user applying an outside force.

10. The safety lancet of claim 1, wherein the puncture device may be any device that can pierce the atrial septum and withdraw.

11. A safety lancet transseptal access system comprising:
a housing having a proximal end and a distal end, the proximal end having an opening with a plunger and the distal end terminating in a housing wall, a housing lumen extending through the housing and a release mechanism coupled to the housing;
a puncture device spring connected to the plunger and a disk; and
a tubular dilator having an elongated body having a proximal end portion connected to the housing wall and a distal end portion terminating in a distal tip, a dilator lumen extending through the body, the body being dimensioned to slidingly receive a hypotube and a puncture device, the puncture device being connected to the disk;
wherein when the plunger is pushed towards the distal end of the housing, the puncture device spring compresses triggering the release mechanism which then causes the puncture device to be deployed past the housing wall, and
wherein once deployed, tension from the puncture device spring pulls the disk and the puncture device back into the housing to a neutral position.

12. The safety lancet of claim 11, wherein the housing further comprises a plunger return spring and wherein the plunger return spring is configured to pull the plunger back to an undeployed state once deployed.

13. The safety lancet of claim 11, wherein the puncture device and the hypotube may be redeployed.

14. The safety lancet of claim 11, wherein the hypotube may be extended out of the distal tip of the dilator by the puncture device spring.

15. The safety lancet of claim 11, wherein the puncture device is an automatic spring-loaded puncture device.

16. The safety lancet of claim 11, wherein the puncture device is mechanically triggered by a user by applying an outside force.

17. The safety lancet of claim 11, wherein the puncture device may be any device that can pierce the atrial septum and withdraw.

18. A method of making a safety lancet transseptal access system, the method comprising:
providing a housing having a proximal end and a distal end, the proximal end having an opening with a plunger and the distal end terminating in a housing wall, a housing lumen extending through the housing and a release mechanism coupled to the housing;
securing a puncture device spring connected to the plunger and a disk; and
a puncture device connected to the disk;
wherein when the plunger is pushed towards the distal end of the housing, the puncture device spring compresses triggering the release mechanism which then causes the puncture device to be deployed past the housing wall, and
wherein once deployed, tension from the puncture device spring pulls the disk and the puncture device back into the housing to a neutral position.

19. The safety lancet of claim 18, wherein the housing further comprises a plunger return spring.

20. The safety lancet of claim 19, wherein the puncture device may be redeployed.
